(19) United States
(12) Patent Application Publication
(10) Pub. No.: US 2025/0276176 A1
(43) Pub. Date: Sep. 4, 2025

(54) APPARATUS AND METHODS FOR APPLYING AN ELECTRICAL SIGNAL TO A SUBJECT

(71) Applicant: THERANICA BIO-ELECTRONICS LTD., Netanya (IL)

(72) Inventors: Alon IRONI, Haifa (IL); Ronen JASHEK, Shoham (IL); Ofer RIVKIND BARLEV, Modi’in (IL); Liron RABANY, Herzliya (IL)

(73) Assignee: THERANICA BIO-ELECTRONICS LTD., Netanya (IL)

(21) Appl. No.: 19/069,327

(22) Filed: Mar. 4, 2025

Related U.S. Application Data
(60) Provisional application No. 63/560,904, filed on Mar. 4, 2024.

(51) Int. Cl. A61N 1/36; A61N 1/04

(52) U.S. Cl. CPC A61N 1/36034; A61N 1/0452; A61N 1/0492; A61N 1/36021; A61N 1/36031

(57) ABSTRACT

An apparatus includes a computer processor configured to drive electrodes to apply an amplitude shift keying signal into a portion of a subject's body. The amplitude shift keying signal contains a high frequency component that acts as a carrier wave, and a low frequency component that acts as a modulating component that modulates the carrier wave. The low frequency signal is applied such that when a pulse of the low frequency signal is active, a current of the amplitude shift keying signal alternates between a nominal maximum and a nominal minimum of the amplitude shift keying signal; and when a pulse of the low frequency signal is inactive, the current of the amplitude shift keying signal alternates between the nominal maximum minus a modulation factor and the nominal minimum plus the modulation factor, the modulation factor being between 0.05 and 0.15 of the nominal maximum. Other embodiments are also described.

### CROSS-REFERENCE TO RELATED APPLICATIONS

The present application claims the benefit of U.S. Provisional Application 63/560,904, filed Mar. 4, 2024, which is assigned to the assignee of the present application and incorporated herein by reference.

### FIELD OF EMBODIMENTS OF THE INVENTION

Some applications of the present invention generally relate to medical apparatus and methods. Specifically, some applications of the present invention relate to apparatus and methods for electrical stimulation of a subject's body.

### BACKGROUND

U.S. Pat. No. 11,357,980 to Ironi et al., which is incorporated herein by reference, describes placing a set of two or more electrodes in electrical contact with a portion of a subject's body. A computer processor drives the electrodes to apply an amplitude shift keying signal to the portion of the subject's body, the amplitude shift keying signal containing a high frequency component that acts as a carrier wave, the high frequency component having a frequency of between 80 Hz and 120 Hz, and a low frequency component that acts as a modulating component that modulates the carrier wave, the low frequency component having a frequency of between 1 Hz and 8 Hz. Other applications are also described.

WO 2018/215879 to Ironi, which is incorporated herein by reference, describes electrodes configured to be placed upon a portion of a body of a subject, and a user interface device. A computer processor applies a neuromodulation treatment to the subject, by driving electrical pulses into the portion of the subject's body via the electrodes, and generates an output on the user interface device that indicates to the subject a physiological effect that the neuromodulation treatment has upon the subject's body. Other applications are also described.

Fibromyalgia is a chronic disorder that causes pain and tenderness throughout the body, as well as fatigue and trouble sleeping.

The following references may be of interest:
U.S. Pat. No. 6,839,594 to Cohen et al.
U.S. Pat. No. 8,774,925 to Yarnitsky
US 2006/0047325 to Thimineur et al.
US 2006/0149337 to John
WO 2016/155773 to Muller et al.
WO 2019/138407 to Jashek et al.
Wu P, Zhu L, Zheng S Y, et al. Transcutaneous Electrical Acupoint Stimulation for Moderate to Severe Pain in Hepatocellular Carcinoma: A Protocol for a Randomized Controlled Trial. J Pain Res. 2022; 15:1889-1896. doi: 10.2147/JPR.S361821
Han J S, Chen X H, Sun S L, et al. Effect of low- and high-frequency TENS on Met-enkephalin-Arg-Phe and dynorphin A immunoreactivity in human lumbar CSF. Pain. 1991; 47 (3): 295-298. doi: 10.1016/0304-3959 (91) 90218-M
Alsouhibani A, Hoeger Bement M. Impaired conditioned pain modulation was restored after a single exercise session in individuals with and without fibromyalgia. Pain Rep. 2022; 7 (3): e996. doi: 10.1097/PR9.0000000000000996
I H Jonsdottir 1, P Hoffmann, P Thorèn. Physical exercise, endogenous opioids and immune function. Acta Physiol Scand Suppl. 1997; 640:47-50.
Koltyn K et al. Perception of pain following aerobic exercise Med Sci Sports Exerc 1996 November; 28 (11): 1418-21.
Bidari A, Ghavidel-Parsa B, Rajabi S, Sanaei O, Toutounchi M. The acute effect of maximal exercise on plasma beta-endorphin levels in fibromyalgia patients. Korean J Pain. 2016; 29 (4): 249-254. doi: 10.3344/kjp.2016.29.4.249
Sarmento C V M, Liu Z, Smirnova I V, Liu W. Exploring Adherence to Moderate to High-Intensity Exercises in Patients with Fibromyalgia: The Role of Physiological and Psychological Factors—A Narrative Literature Review. Physiologia. 2023; 3 (3): 472-483. doi: 10.3390/physiologia3030034
Salazar A P de S, Stein C, Marchese R R, Plentz R D M, Pagnussat A D S. Electric Stimulation for Pain Relief in Patients with Fibromyalgia: A Systematic Review and Meta-analysis of Randomized Controlled Trials. Pain Physician. 2017; 20(2): 15-25
O'Brien A T, Deitos A, Triñanes Pego Y, Fregni F, Carrillo-de-la-Peña M T. Defective Endogenous Pain Modulation in Fibromyalgia: A Meta-Analysis of Temporal Summation and Conditioned Pain Modulation Paradigms. J Pain. 2018 August; 19 (8): 819-836. Doi: 10.1016/j.jpain.2018.01.010. Epub 2018 Feb. 15. PMID: 29454976
Pin Wu et al. Transcutaneous Electrical Acupoint Stimulation for Moderate to Severe Pain in Hepatocellular Carcinoma: A Protocol for a Randomized Controlled Trial. J Pain Res. 2022; 15:1889-1896. Published online 2022 Jul. 6. Doi: 10.2147/JPR.S361821
Flora, R. et al. β-endorphin response to aerobic and anaerobic exercises in Wistar male rats Medical Journal of Indonesia. Vol 29 No 3 (2020): September.
Gozani S. Journal of Pain Research 2019 (12). Remote Analgesic Effects Of Conventional Transcutaneous Electrical Nerve Stimulation: A Scientific And Clinical Review With A Focus On Chronic Pain.

### SUMMARY OF THE APPLICATION

The inventors have noted that exercise stimulates the secretion of endorphins and enkephalins (see Jonsdottir et al., cited above), and is associated with reduced pain perception and a higher pain threshold (see Koltyn K et al., cited above). Exercise is known as an effective intervention in fibromyalgia treatment (see Bidari et al., cited above). However, many patients are unable to implement this recommendation due to physiological and psychological factors associated with fibromyalgia (see Sarmento et al., cited above). Studies have shown that low frequency (2 Hz) electric stimulation causes significant elevation of met-enkephalin (see Han et al., cited above), as well as endorphins (see Wu et al., cited above). Electric muscle stimulation was found to have a positive effect on pain in fibromyalgia patients (see Salazar et al., cited above). This analysis serves as a basis for the low-frequency component of the amplitude shift keying signal provided by some applications of the present invention.

For some applications, apparatus and methods described herein are used to treat fibromyalgia. Typically, in response to the subject experiencing symptoms of fibromyalgia in a first anatomical region, electrodes are placed on a second anatomical region of the subject body (which is different from the first anatomical region). Electrical energy is applied to the second anatomical region by driving electrical pulses into the second anatomical region, thereby typically stimulating motor neurons that innervate muscles in the anatomical region. For some applications, the electrodes are placed at a location that is at a distance of more than 25 cm from the location at which the subject is experiencing pain, and the electrical energy is applied to the location at which the electrodes are placed. Typically, by applying electrical energy at the second anatomical region, pain at the first anatomical region is reduced, e.g., at least in part via the conditioned pain modulation mechanism, which may increase levels of beta-endorphins in the cerebrospinal fluid, resulting in reduced pain perception.

In accordance with some applications of the present invention, a computer processor drives electrodes to apply electrical stimulation pulses to the subject's body, such that substantially for the duration of the application of the neurostimulation (e.g., more than 90 percent of the time that the neurostimulation is being applied) the signal that is being applied contains both a high frequency component and a low frequency component. Typically, the signal that is applied is an amplitude shift keying signal, with the high frequency component acting as a carrier wave, and the low frequency component acting as a modulating wave that modulates the carrier wave. The computer processor typically configures the low frequency component such that: (a) when a pulse of the low frequency component is active, a current of the amplitude shift keying signal alternates between a nominal maximum and a nominal minimum of the amplitude shift keying signal, and (b) when a pulse of the low frequency component is inactive, the current of the amplitude shift keying signal alternates between the nominal maximum minus a modulation factor and the nominal minimum, the modulation factor being between 0.05 and 0.15 of the nominal maximum.

For some applications, the high frequency component has a frequency of more than 80 Hz (e.g., more than 90 Hz), and/or less than 120 Hz (e.g., less than 110 Hz), e.g., between 80 Hz and 120 Hz, or between 90 Hz and 110 Hz. For some applications, the low frequency component has a frequency of more than 1 Hz (e.g., more than 1.5 Hz), and/or less than 8 Hz (e.g., less than 4 Hz), e.g., between 1 Hz and 8 Hz, or between 1.5 Hz and 4 Hz.

There is therefore provided, in accordance with an Inventive Concept 1 of the present invention, an apparatus including:
a set of two or more electrodes configured to be placed in electrical contact with a portion of a body of a subject; and
at least one computer processor configured to drive the electrodes to apply an amplitude shift keying signal into the portion of the subject's body, the amplitude shift keying signal containing:
   a high frequency component that acts as a carrier wave, the high frequency component having a frequency of between 80 Hz and 120 Hz, and
   a low frequency component that acts as a modulating component that modulates the carrier wave, the low frequency component having a frequency of between 1 Hz and 8 Hz,
the computer processor configured to drive the electrodes to apply the amplitude shift keying signal into the portion of the subject's body by applying the low frequency signal such that:
   when a pulse of the low frequency signal is active, a current of the amplitude shift keying signal alternates between a nominal maximum and a nominal minimum of the amplitude shift keying signal, and
   when a pulse of the low frequency signal is inactive, the current of the amplitude shift keying signal alternates between the nominal maximum minus a modulation factor and the nominal minimum plus the modulation factor, the modulation factor being between 0.05 and 0.15 of the nominal maximum.
Inventive Concept 2. The apparatus according to Inventive Concept 1, wherein the computer processor is configured to set the high frequency component to be between 100 Hz and 120 Hz.
Inventive Concept 3. The apparatus according to any one of Inventive Concepts 1-2, further including an accelerometer configured to generate an accelerometry signal indicative of motion of the portion of the body of the subject, wherein the computer processor is configured to modify a parameter of the amplitude shift keying signal in response to the accelerometry signal.
Inventive Concept 4. The apparatus according to Inventive Concept 3, wherein the accelerometer is configured to generate the accelerometry signal in response to contraction of a muscle of the subject.
Inventive Concept 5. The apparatus according to Inventive Concept 4, wherein the computer processor is configured to increase an energy level of the amplitude shift keying signal in response to determining that a level of the accelerometry signal is below a threshold.
Inventive Concept 6. The apparatus according to Inventive Concept 4, wherein the accelerometry signal is indicative of cyclic contraction of the muscle, and wherein the computer processor is configured to increase an energy level of the amplitude shift keying signal in response to determining that a level of the accelerometry signal is below a threshold.
Inventive Concept 7. The apparatus according to any one of Inventive Concepts 1-2, further including an accelerometer configured to generate an accelerometry signal indicative of motion of the portion of the body of the subject, wherein the computer processor is configured to direct the subject to move the set of two or more electrodes in response to a low level of the accelerometry signal.
Inventive Concept 8. The apparatus according to any one of Inventive Concepts 1-2,
   wherein the set of two or more electrodes is a first set of two or more electrodes,
   wherein the apparatus further includes a second set of two or more electrodes, and an accelerometer configured to generate an accelerometry signal indicative of motion of the portion of the body of the subject, and
   wherein the computer processor is configured to drive the electrodes of the second set, rather than the electrodes of the first set, to apply the amplitude shift keying signal in response to a low level of the accelerometry signal.
Inventive Concept 9. The apparatus according to any one of Inventive Concepts 1-2, further including a patch, wherein the electrodes are disposed upon the patch and the electrodes are configured to be placed in electrical contact with the portion of the subject's body by placing the patch upon the portion of the subject's body.
Inventive Concept 10. The apparatus according to any one of Inventive Concepts 1-2, further including a mount selected from the group consisting of: a wristwatch, a cuff, and a bracelet, and wherein the electrodes are disposed upon the selected mount and are configured to be placed in electrical contact with the portion of the subject's body by placing the selected mount upon the portion of the subject's body.
Inventive Concept 11. The apparatus according to any one of Inventive Concepts 1-2, wherein the computer processor is configured to drive the electrodes to apply the amplitude shift keying signal into the portion of the subject's body by:
   applying the high frequency component, the high frequency component including a biphasic pulse, and
   applying the low frequency component, the low frequency component including a monophasic pulse.
Inventive Concept 12. The apparatus according to any one of Inventive Concepts 1-2, wherein the computer processor is configured to drive the electrodes to apply the amplitude shift keying signal into the portion of the subject's body by applying the high frequency component, the high frequency component having a base frequency, and the frequency of the high frequency component drifting from the base frequency up to 20 percent above the base frequency, and down to 20 percent below the base frequency.

There is further provided, in accordance with an Inventive Concept 13 of the present invention, a method including:
applying an electrical amplitude shift keying signal to a portion of a body of a subject, via electrodes, the amplitude shift keying signal containing:
   a high frequency component that acts as a carrier wave, the high frequency component having a frequency of between 80 Hz and 120 Hz, and
   a low frequency component that acts as a modulating component that modulates the carrier wave, the low frequency component having a frequency of between 1 Hz and 8 Hz,
wherein applying the electrical amplitude shift keying signal to the portion of the subject's body includes applying the low frequency signal such that:
   when a pulse of the low frequency signal is active, a current of the amplitude shift keying signal alternates between a nominal maximum and a nominal minimum of the amplitude shift keying signal, and
   when a pulse of the low frequency signal is inactive, the current of the amplitude shift keying signal alternates between the nominal maximum minus a modulation factor and the nominal minimum plus the modulation factor, the modulation factor being between 0.05 and 0.15 of the nominal maximum.
Inventive Concept 14. The method according to Inventive Concept 13, wherein applying the electrical amplitude shift keying signal includes applying the electrical amplitude shift keying signal to a portion of a body of a subject who has fibromyalgia.
Inventive Concept 15. The method according to any one of Inventive Concepts 13-14, wherein applying the electrical amplitude shift keying signal to the portion of the subject's body includes:
   applying the high frequency component, the high frequency component including a biphasic pulse, and
   applying the low frequency component, the low frequency component including a monophasic pulse.
Inventive Concept 16. The method according to any one of Inventive Concepts 13-14, wherein applying the electrical amplitude shift keying signal to the portion of the subject's body includes applying the high frequency component, the high frequency component having a base frequency, and the frequency of the high frequency component drifting from the base frequency up to 20 percent above the base frequency, and down to 20 percent below the base frequency.
Inventive Concept 17. The method according to Inventive Concept 13, wherein applying the electrical amplitude shift keying signal includes setting the high frequency component to be between 100 Hz and 120 Hz.
Inventive Concept 18. The method according to Inventive Concept 13, wherein applying the electrical amplitude shift keying signal further includes modifying a parameter of the amplitude shift keying signal in response to an accelerometry signal generated by an accelerometer and indicative of motion of the portion of the body of the subject.
Inventive Concept 19. The method according to Inventive Concept 18, wherein the accelerometer is configured to generate the accelerometry signal in response to contraction of a muscle of the subject.
Inventive Concept 20. The method according to Inventive Concept 19, wherein applying the electrical amplitude shift keying signal includes increasing an energy level of the amplitude shift keying signal in response to determining that a level of the accelerometry signal is below a threshold.
Inventive Concept 21. The method according to Inventive Concept 19, wherein the accelerometry signal is indicative of cyclic contraction of the muscle, and wherein applying the electrical amplitude shift keying signal includes increasing an energy level of the amplitude shift keying signal in response to determining that a level of the accelerometry signal is below a threshold.
Inventive Concept 22. The method according to Inventive Concept 13, further including directing the subject to move the electrodes in response to a low level of an accelerometry signal generated by an accelerometer and indicative of motion of the portion of the body of the subject.
Inventive Concept 23. The method according to Inventive Concept 13,
   wherein the electrodes are first electrodes, and
   wherein applying the electrical amplitude shift keying signal includes applying the amplitude shift keying signal via second electrodes, rather than the first electrodes, in response to a low level of an accelerometry signal generated by an accelerometer and indicative of motion of the portion of the body of the subject.

There is still further provided, in accordance an Inventive Concept 24 of the present invention, a method including:
applying an electrical amplitude shift keying signal to a portion of a body of a subject who has fibromyalgia, via electrodes, the amplitude shift keying signal containing:
   a high frequency component that acts as a carrier wave, the high frequency component having a frequency of between 80 Hz and 120 Hz, and
   a low frequency component that acts as a modulating component that modulates the carrier wave, the low frequency component having a frequency of between 1 Hz and 8 Hz.
Inventive Concept 25. The method according to Inventive Concept 24, wherein applying the electrical amplitude shift keying signal includes setting the high frequency component to be between 100 Hz and 120 Hz.

The present invention will be more fully understood from the following detailed description of embodiments thereof, taken together with the drawings, in which:

### BRIEF DESCRIPTION OF THE DRAWINGS

FIG. 1 is a schematic illustration of a patch having electrodes disposed thereon, a computer processor, and a user interface, in accordance with some applications of the present invention;

FIG. 2A is a representation of a high frequency component of an electrical stimulation signal that is applied to a subject, in accordance with some applications of the present invention;

FIG. 2B is a representation of a single monophasic pulse of a high frequency component of an electrical stimulation signal that is applied to a subject, in accordance with some applications of the present invention;

FIG. 3A is a representation of a low frequency component of an electrical stimulation signal that is applied to a subject, in accordance with some applications of the present invention;

FIG. 3B which is a representation of a single monophasic pulse of a low frequency component of an electrical stimulation signal that is applied to a subject, in accordance with some applications of the present invention; and

FIG. 4 shows an electrical stimulation signal that includes a high frequency component and a low frequency component that is applied to a portion of a subject's body, in accordance with some applications of the present invention.

### DETAILED DESCRIPTION OF APPLICATIONS

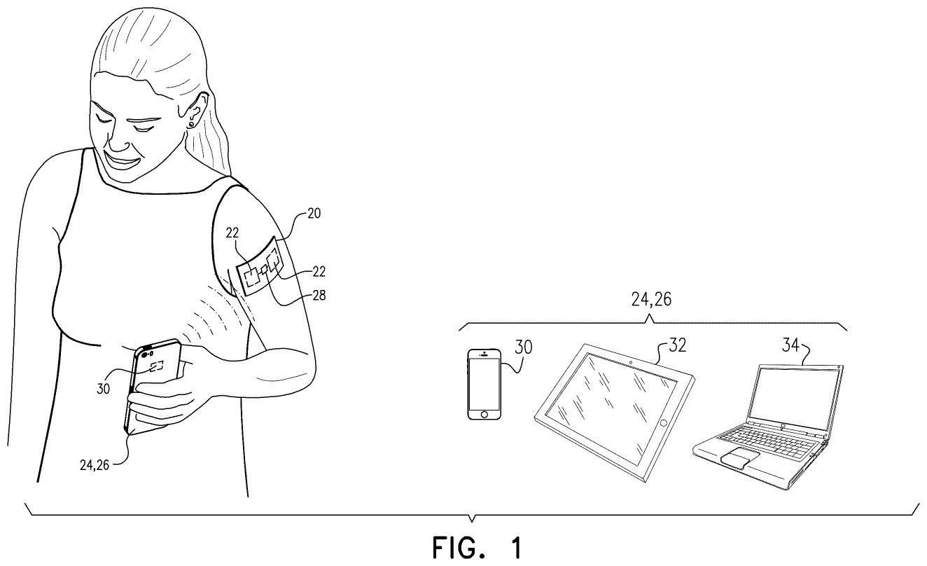

Reference is now made to FIG. 1, which is a schematic illustration of a mount, such as a patch 20, that supports electrodes 22 disposed on a subject's arm (e.g., upper arm) or leg (e.g., upper or lower leg), a computer processor 24, and a user interface 26, in accordance with some applications of the present invention. For some applications, the apparatus and methods described herein are used to treat fibromyalgia.

It is noted that the particular positioning of electrodes 22 on the subject's arm as shown in FIG. 1 is by way of illustration and not limitation. The scope of the present invention includes placing the electrodes on another site of the subject's body, e.g., elsewhere on the arm, e.g., on the wrist. As appropriate based on the site, the physical form of patch 20 may be suitably altered. For example, if electrodes 22 are to be placed on the wrist, then they may be incorporated into a cuff, wristwatch or bracelet type of mount. Thus, in all instances in the present application where a patch is described or shown, other suitable mounts for electrodes 22 (such as a wristwatch or bracelet) may be used depending on where or how the electrodes are to be placed.

Typically, as part of a scheduled regimen to treat symptoms of fibromyalgia in a first anatomical region, or in response to the subject experiencing or anticipating symptoms of fibromyalgia in the first anatomical region, the electrodes are placed on a second anatomical region of the subject body (which is different from the first anatomical region). Electrical energy is applied to the second anatomical region by driving electrical pulses into the second anatomical region. For some applications, the electrodes are placed at a location that is at a distance of more than 25 cm from the location at which the subject is experiencing pain, and the electrical energy is applied to the location at which the electrodes are placed. Typically, by applying electrical energy at the second anatomical region, a symptom of fibromyalgia at the first anatomical region is reduced, e.g., at least in part via the conditioned pain modulation mechanism.

For some applications, transcutaneous electrical energy is applied caudally to the neck of the subject using electrodes 22 disposed on patch 20. For some applications, as part of a scheduled regimen to treatment symptoms of fibromyalgia or in response to the subject experiencing or anticipating symptoms of fibromyalgia, the subject places patch 20 upon a part of the subject's body, such as the subject's upper arm, as shown in FIG. 1. For some applications, rather than placing a patch on the subject, the subject wears a cuff, sleeve, or wrap having a plurality of electrodes 22 coupled thereto. For some applications, the electrodes are placed on a different portion of the subject's body, such as a different location on the subject's arm, on the subject's hands, legs, feet, and/or lower abdomen. Typically, the electrodes are placed in electrical contact with the subject's skin. Further typically, an electronics module 28 contained within the patch controls the electrodes, in response to control signals, which may be wiredly or wirelessly received from the computer processor.

For some applications, user interface 26 includes user interface components of one or more devices, such as a smartphone 30, a tablet device 32, and/or a personal computer 34. Typically, for such applications, computer processor 24 is the computer processor of the device. It is noted that, although FIG. 1 shows the user using a smartphone as the user interface and the computer processor, the scope of the present application includes using other devices for this purpose, e.g., tablet device 32, or personal computer 34. For some applications, electronics module 28 performs some of the computer processor functionalities that are described herein. Alternatively or additionally, the electronics module is used to facilitate communication between a computer processor of an external device (such as smartphone 30, tablet device 32, and/or personal computer 34) and the electrodes, typically using known protocols, such as Wifi, Bluetooth®, ZigBee®, or any near field communication (NFC) protocol. Alternatively, the computer processor, the user interface, and all related electronics are disposed on patch 22. These arrangements allow the subject to walk around and to continue to engage in regular activities of daily living during a treatment session.

Electronics module 28 typically comprises a power source, a central processing unit (CPU), typically programmed in microcode, that controls the electrodes, one or more memory units for storing the stimulation sequences during the stimulation, an impulse generator, and components for wireless communication. For some applications, the electronics module is an integrated system-on-chip (SoC). The electronics module typically includes electronic circuitry, which, by way of example, may include components such as diodes, resistors, and capacitors, etc.

For some applications, the computer processor receives an input from the subject that indicates that the subject is experiencing symptoms of fibromyalgia or is ready to initiate a scheduled treatment regimen, via a program or application that is run on the computer processor (e.g., a program or application that is run on smartphone 30, tablet device 32, and/or personal computer 34). In response to the input, the computer processor communicates a control signal to the electronics module. Typically, in response to receiving the control signal, the electronics module drives the electrodes to drive electrical stimulation pulses into the subject (e.g., into the subject's upper arm, as shown in FIG. 1). For some applications, the computer processor receives an input from the subject indicating a particular treatment program, and/or control stimulation parameters (such as the intensity of the stimulation) that should be provided.

For some applications, the computer processor is configured to drive the electrodes to provide stimulation to the subject to prevent the onset of symptoms of fibromyalgia or ameliorate the symptoms of fibromyalgia, before such events are sensed by the subject. A fibromyalgia attack, often called a flare-up, is a sudden worsening of symptoms that can last days, weeks, or even longer. Unlike acute pain conditions where pain has a clear start and stop, fibromyalgia flares are unpredictable, prolonged, and multifaceted. Fibromyalgia flare-ups can persist for days or even longer. Therefore, a preventive approach is beneficial for the patient. For example, a stimulation treatment as described herein may be delivered at regular intervals, e.g., daily or twice daily, e.g., for 15-30 minutes or 30-60 minutes per session, e.g., up to 45 minutes per session. In accordance with respective applications, the computer processor (via a program or application running on the processor) may facilitate the scheduling of such treatments, and/or may automatically alert the subject when necessary, in order to facilitate compliance with the treatment schedule.

In accordance with some applications of the present invention, the computer processor drives the electrodes to apply electrical stimulation pulses to the subject's body, such that substantially for the duration of the application of the neurostimulation (e.g., more than 90 percent of the time that that the neurostimulation is being applied) the signal that is being applied contains both a high frequency component and a low frequency component. Typically, the signal that is applied is an amplitude shift keying signal, with the high frequency component acting as a carrier wave, and the low frequency component acting as a modulating wave that modulates the carrier wave. For some applications, the high frequency component has a frequency of more than 80 Hz (e.g., more than 90 Hz), and/or less than 120 Hz (e.g., less than 110 Hz), e.g., between 80 Hz and 120 Hz, or between 90 Hz and 110 Hz. For some applications, the low frequency component has a frequency of more than 1 Hz (e.g., more than 1.5 Hz), and/or less than 8 Hz (e.g., less than 4 Hz), e.g., between 1 Hz and 8 Hz, or between 1.5 Hz and 4 Hz, e.g., 2.5 Hz.

Optionally, the modulated signal is a symmetrical bi-phasic rectangular pulse.

Both the high and low frequency components of the electrical simulation typically stimulate descending analgesic mechanisms. For some applications, the low frequency component primarily stimulates endorphin and/or enkephalin release, while the high frequency component primarily stimulates serotonin and/or noradrenaline (norepinephrine) release.

More specifically, for some applications, the high frequency component may provide Conditioned Pain Modulation (CPM), which is an endogenous mechanism for pain alleviation. In CPM, peripheral activation of C fibers and A delta fibers leads to activation of the central nervous system, which causes the release of pain-relieving neurotransmitters norepinephrine and serotonin. The low frequency component may promote the release of beta endorphins and/or Metenkephalin-Arg-Phe (MEAP), such as when applied to different muscles (e.g., major muscles).

The techniques described herein may be effective in the treatment of fibromyalgia because studies show a strong connection between sustained cyclic contraction of muscles and the release of beta-endorphins and enkephalins. Beta-endorphins are peptides with a significant effect on pain relief and mood improvement. There is a known relationship between physical activity and the release of beta-endorphins and the sensation of euphoria after completing a workout, including among individuals suffering from fibromyalgia. Enkephalins are peptides associated with reducing stress, anxiety, and the perception of pain.

As provided herein, the release of beta-endorphins and enkephalins can be encouraged by electrical stimulation of muscle. The activation mechanism is related to the contraction and relaxation of the muscle over time (tens of minutes). These contractions and relaxations can be produced by stimulation at a low frequency, optionally with high intensity.

For some applications, larger muscles are activated, as there may be a direct correlation between the size of the muscle and the amount of endorphins and enkephalins released. As provided herein, it is possible to combine electrical stimulation with muscle contraction (low frequency) and the stimulation of C fibers to activate CPM without them interfering with each other or cannibalizing the response of the other.

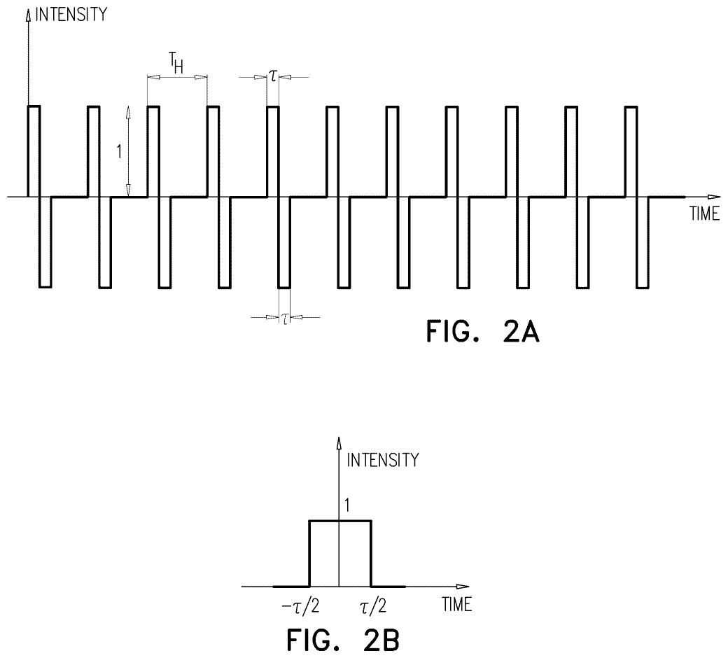

Reference is now made to FIG. 2A, which is a representation of a high frequency component of an electrical stimulation signal that is applied to a subject, in accordance with some applications of the present invention. Reference is also made to FIG. 2B, which is a representation of a single monophasic pulse of a high frequency component of an electrical stimulation signal that is applied to a subject, in accordance with some applications of the present invention.

Typically, the high frequency component is a train (typically, an effectively infinite train) of biphasic or monophasic pulses. For some applications, the pulses are periodic rectangular waves. For example, as shown in FIG. 2A, the high frequency component of the signal has a period T_{H}, each period including a portion during which the signal is inactive and a portion during which a biphasic pulse is applied, the biphasic pulse having a pulse duration of 2τ. Typically, a frequency f_{H} of the high frequency component of the signal (which is the inverse of T_{H}) is more than 80 Hz (e.g., more than 90 Hz), and/or less than 120 Hz (e.g., less than 110 Hz), e.g., between 80 Hz and 120 Hz, or between 90 Hz and 110 Hz.

FIG. 2B is a representation of a single monophasic pulse of a high frequency component of an electrical stimulation signal that is applied to a subject, in accordance with some applications of the present invention. It is noted that, for some applications, a biphasic signal is applied (as shown in FIG. 2A), in which case the signal is composed of two such rectangles, one in the positive direction and the other in the negative direction. For some applications, a biphasic signal rather than a monophasic signal is applied, in order that the total electrical charge delivered to the body should be substantially zero.

The basic monophasic pulse (“rect(t)”) is defined as follows:

$$rect ( t ) = \{ \begin{matrix}1 & \vert t \vert \leq τ / 2 \\ 0 & \vert t \vert > τ / 2\end{matrix}$$

where τ is the temporal width of the pulse. For some applications, the temporal width is more than 50 microseconds (e.g., more than 80 microseconds), and/or less than 300 microseconds (e.g., less than 250 microseconds), e.g., between 50 microseconds and 300 microseconds, or between 150 microseconds and 250 microseconds. The amplitude is denoted “1,” this value representing the nominal amplitude of the current.

The complete waveform of the high frequency component c(t), may be described as follows:

$$c ( t ) = {\sum }_{n = 0}^{N - 1} ( rect ( t + \frac{τ}{2} - {nT}_{H} ) - rect ( t - \frac{τ}{2} - {nT}_{H} ) )$$

where N is the number of periods. Typically, when a treatment is applied using the techniques described herein, the treatment is applied for between 10 minutes and one hour (e.g., between 15 minutes and 30 minutes). Over the treatment period, the train of pulses is effectively infinite, meaning that the value of N is effectively infinity.

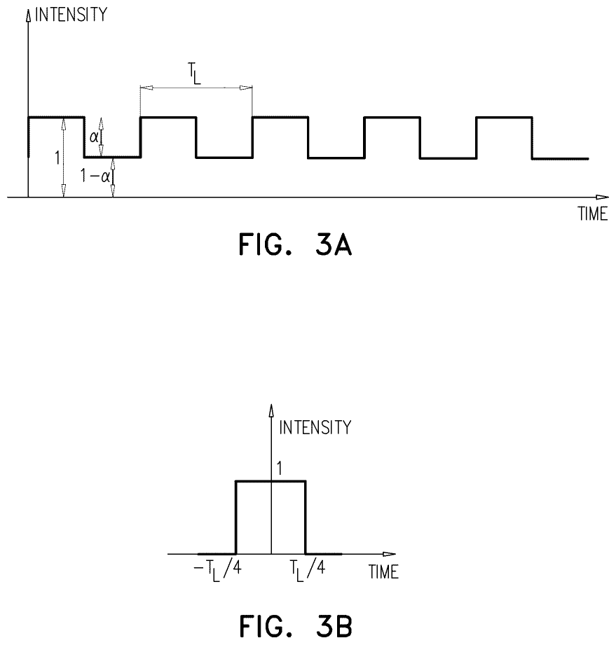

Reference is now made to FIG. 3A, which is a representation of a low frequency component of an electrical stimulation signal that is applied to a subject, in accordance with some applications of the present invention. Reference is also made to FIG. 3B, which is a representation of a single monophasic pulse of a low frequency component of an electrical stimulation signal that is applied to a subject, in accordance with some applications of the present invention.

Typically, the low frequency component is a train (typically, an effectively infinite train) of monophasic pulses. For some applications, the low frequency component has a frequency of more than 1 Hz (e.g., more than 1.5 Hz), and/or less than 8 Hz (e.g., less than 4 Hz), e.g., between 1 Hz and 8 Hz, or between 1.5 Hz and 4 Hz. For some applications, the low frequency signal has a duty cycle of 50 percent. When the pulse is inactive, the current of the low frequency signal is a given percentage (e.g., approximately 50 percent) of the maximal current of the low frequency signal, and when the pulse is active, the current of the low frequency signal is at its maximum.

FIG. 3B is a representation of a single monophasic pulse of a low frequency component of an electrical stimulation signal that is applied to a subject, in accordance with some applications of the present invention. The basic monophasic pulse (“mₒ(t)”) is defined as follows:

$${m}_{0} ( t ) = \{ \begin{matrix}1 , & \vert t \vert \leq {T}_{L} / 4 \\ 0 , & \vert t \vert > {T}_{L} / 4\end{matrix}$$

where T_{L} is the period of the low frequency component of the signal. The amplitude 1 represents the maximum nominal amplitude of the current, which is the current when the low frequency pulse is active. The maximum nominal current is modulated by a modulation factor α, when the pulse is inactive, a being a fraction, i.e., 0<α<1. The use of a symmetric expression for m₀(t) between positive and negative T_{L}/4 in the equation above is by way of illustration and not limitation.

Denoting the frequency of the low frequency component of the signal by f_{L}, the complete waveform of the low frequency component m(t) may be described as follows:

$$m ( t ) = 1 - α + α {\sum }_{n = 0}^{N - I} {m}_{0} ( t - {nT}_{L} )$$

where:
N is the number of periods (which is typically effectively infinity, as described hereinabove).

Typically, the high frequency component acts as a carrier wave, and the low frequency component acts as a modulating wave that modulates the carrier wave. The modulation factor is the factor by which the low frequency wave modulates the high frequency wave, during the inactive phase of the duty cycle of the low frequency signal. In other words, when the pulse of the low frequency signal is active, the overall current of the amplitude shift keying signal alternates between the nominal maximum and the nominal minimum of the amplitude shift keying signal. When the pulse of the low frequency signal is inactive, the current of the amplitude shift keying signal alternates between nominal maximum minus the modulation factor and the nominal minimum plus the modulation factor.

If the modulation factor is small (i.e., close to 0), the impact of the modulating wave will be low, and (vice versa) if the modulating factor is large (i.e., close to 1), the impact of the modulating wave will be high. Typically, the modulating factor is more than 0.05 (e.g., more than 0.08) and/or less than 0.15 (e.g., less than 0.12).

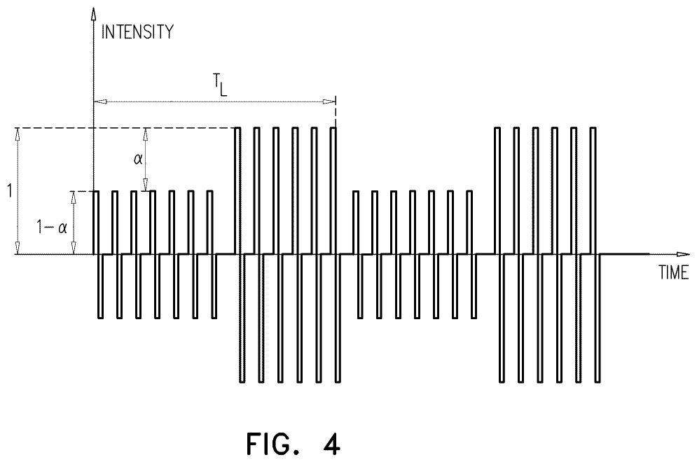

Reference is now made to FIG. 4, which shows an electrical stimulation signal that includes a high frequency component and a low frequency component that is applied to a portion of a subject's body, in accordance with some applications of the present invention. The signal that is applied to the subject is a combination of the high frequency component (i.e., the carrier wave (c(t)), and the low frequency component (i.e., the modulating wave (m(t)). The overall waveform (s(t)) is the two waveforms multiplied by one another, and may be defined as:

s(t)=m(t)c(t)

As may be observed in FIG. 4, the amplitude of the resulting waveform (s(t)):
(a) alternates between +1 and −1 when the pulse of the low frequency signal is active; and
(b) alternates between + (1−α) and −(1−α), when the pulse of the low frequency signal is inactive.

Thus, the subject is simultaneously stimulated with both high frequency stimulation and low frequency stimulation. Both the high and low frequency components of the electrical simulation stimulate descending analgesic mechanisms. For some applications, the low frequency component primarily stimulates endorphin and/or enkephalin release, while the high frequency component primarily stimulates serotonin and/or noradrenaline release.

It is noted that the above discussion regarding the output waveform s(t) is an example of how applications of the present invention may be performed. Alternatively, a different high-frequency signal c′(t) and modulating low-frequency signal m′(t) may be used, in which m′(t) amplifies c′(t) rather than suppressing c′(t).

For some applications, the high frequency component of the signal drifts between 10 percent to 20 percent below its base frequency and 10 percent to 20 percent above its base frequency. For example, starting at its base frequency, the frequency of the high frequency component of the signal may be increased by 2 Hz every minute, until it reaches 10 percent to 20 percent above its base frequency. The frequency of the high frequency component of the signal may then be decreased by 2 Hz every minute, until it reaches 10 percent to 20 percent below its base frequency. Alternatively or additionally, the frequency may be increased or decreased by approximately 10 Hz every 4 to 5 minutes. For some applications, by causing the high frequency component to drift, conditioning or habituation of the subject to the stimulation is reduced. That is to say that the effect of the phenomenon whereby the perceived reception of the brain to a constant stimulus declines over time is reduced.

For some applications, the pulse width is set to more than 80 microseconds (e.g., more than 150 microseconds), and/or less than 500 microseconds (e.g., less than 300 microseconds, such as less than 250 microseconds), e.g., 80-500 microseconds (e.g., 400 microseconds), 150-300 microseconds, or 150-250 microseconds. For some applications, the current intensity is set to more than 20 mA (e.g., more than 40 mA), and/or less than 80 mA (e.g., less than 70 mA), e.g., 20-80 mA, or 40-70 mA, e.g., no more than 40 mA.

For some applications, electrodes are arranged on the patch (or on a different electrode supporting element, such as a cuff, etc.) such that the current density per unit area of the skin is below 3.75 mA/cm\hat{ }2. In this manner, the electrical energy that is applied via the patch generates a touch sensation to the user, but does not generate a substantial amount of local pain at the location at which the patch is placed on the subject's skin.

For some applications, suitable stimulation parameters for a given user are determined interactively by the user, or a caregiver of the user. For example, the user or the caregiver may gradually increase the stimulation intensity (via the user interface) until it is evident that the intensity has reached the motor threshold (e.g., by seeing or feeling muscles activity). The user or the caregiver may then slightly reduce the stimulation intensity.

As described hereinabove, for some applications the user controls the stimulation via a user interface 26, e.g., the user interface of a device, such as a phone, as shown in FIG. 1. For some applications, during a stimulation session, a computer program (e.g., a smartphone application) allows the user to interactively control the dosage of electrical stimulation that is applied at the second anatomical region. For example, for applications in which electrodes 22 are placed on the subject's upper arm (as shown in FIG. 1), for the purpose of treating fibromyalgia, the computer program may prompt the user to increase the stimulation dosage until the user feels that the stimulation is relieving fibromyalgia symptoms. The computer program may also allow the user to indicate that the user is feeling local pain at the electrode placement location and/or excessive undesired movement. (A certain level of movement due to muscle contractions is acceptable.) In response to such an input from the user, the computer processor may prompt the user to decrease the stimulation dosage until the local pain or undesired movement has stopped. In this manner, via the computer processor of the user interface device, the user is able to interactively control the stimulation such that (a) the fibromyalgia is relieved, but (b) the electrical simulation does not cause local pain at the local stimulation site and/or undesired movement.

For some applications, a sensing pair of electrodes 36 are used. Typically, the sensing electrodes are placed on the skin near the location of the stimulating electrodes. For example, the sensing electrodes may be disposed on patch 20, as shown. The sensing electrodes are typically surface EMG electrodes and are configured to sense the EMG signal generated by motor nerves that enervate a muscle located in the vicinity of the electrodes 22 (e.g., under and in between the pair of electrodes 22). For some applications, the sensing electrodes are configured to sense the EMG signal generated by motor nerves that traverse a location in the vicinity of electrodes 22, but that enervate a muscle located elsewhere (e.g., motor nerves passing through the upper arm towards the hand).

For some applications, in response to the signal sensed by electrodes 36, the computer processor determines changes in the energy of the EMG of the above-described motor nerves. For example, in response to the subject starting, or attempting to move a limb upon which the electrodes are placed, the computer processor detects an increase in the EMG energy. In response thereto, the computer processor reduces the stimulation dose of the electrical stimulation that is delivered via electrodes 22.

(This is because the increase in the EMG energy indicates that the subject is moving or attempting to move his/her limb, and that the electrical stimulation signal may interfere with the movement or attempted movement.) Subsequently, in response to detecting that EMG energy has decreased to a given level for a given time period, the processor automatically increases the stimulation dose of the electrical stimulation. (This is because the decrease in the EMG energy indicates that the subject has stopped moving or attempting to move his/her limb.)

If the following denotation symbols are used:
E_{EMG}=momentary energy of the EMG as measured and calculated by the computer processor;
E_{TH}=a threshold level of EMG energy which the computer processor is configured to interpret as being indicative of limb movement or attempted movement (i.e., a movement threshold level);
E_{HYS}=a difference of EMG energy which the computer processor is configured to interpret as hysteresis when the limb is changing from motion to no motion status;
D_{BASE}=normal stimulation dose, when there is no limb motion, and therefore no need to reduce the dose;
D_{MOTION}=a reduced stimulation dose, to which to system adjusts in case of limb motion detection;
for some applications, the computer processor applies the following algorithm:

If E_{EMG}<E_{TH},D=D_{BASE} (I.e., normal operation, when there is no motion)

If E_{EMG}>=E_{TH},D=D_{MOTION}, (I.e., in case of motion detection, the stimulation dose is reduced.)

Subsequent to the dose having been reduced,

if E_{EMG}<(E_{TH}−E_{HYS}),D=D_{BASE}

(I.e., subsequent to motion, the stimulation dose is re-increased only if the EMG signal drops below the movement threshold level minus an amount of energy that varies with time according to a hysteresis curve.)

For some applications, the values of E_{TH} and E_{HYS} are determined in an individual way for each subject. For example, initially, the subject may calibrate the computer processor, during a calibration phase, using the following technique: The stimulation dose is manually adjusted until the subject is able to feel the stimulation, but the stimulation is not painful. The subject then deliberately performs a few movements with the limb, to let the computer processor record the EMG energy changes that the subject undergoes during changes from still to motion, and from motion to still.

As described hereinabove, for some applications, after the end of motion, the stimulation dose is re-increased only if the EMG signal drops below the movement threshold value minus an amount of energy that varies with time according to a hysteresis curve. The reason for subtracting the value that varies according to a hysteresis curve is to prevent the computer processor from jumping between the normal and reduced stimulation doses, as the detected EMG passes above and below the movement threshold level. This is because the computer processor jumping between the normal and reduced stimulation doses might result in unpleasant sensation for the subject.

For some applications, the value of D_{BASE} is determined based upon the stimulation parameters that the subject selects during the calibration phase, as described hereinabove. The value of D_{MOTION} is typically a given percentage of D_{BASE}, e.g., between 50 and 90 percent, or between 60 and 80 percent, of D_{BASE}.

For some applications, D_{BASE} is initially set as (D_{MOTOR}−ε), where D_{MOTOR} is the threshold for motor nerve activation, and ε is a margin used to ensure that motor activation is avoided. Typically, dose adjustment (e.g., reduction of the electrical stimulation dose during limb motion) is performed by means of intensity adjustment. For some applications, pulse width and/or pulse frequency are adjusted. For some applications, the computer processor determines which of the parameters to adjust in order to perform dose adjustment, by initially adjusting each of the parameters, and determining the adjustment of which of the parameters leads to the lowest dose required for motor activation. The computer processor interprets this as indicating to which of the parameters the subject's neural system has greatest sensitivity, and varies the dose by adjusting this parameter.

For some applications, as an alternative to, or in addition to the computer processor automatically determining that the subject is moving or attempting to move a limb upon which the electrodes are disposed, the subject may provide an input to the computer processor indicating that he/she is moving or attempting to move the limb. Similarly, as an alternative to, or in addition to the computer processor automatically determining that the subject has finished moving or attempting to move the limb, the subject may provide an input to the computer processor indicating that he/she has finished moving or attempting to move the limb.

In some applications of the present invention, electrodes 22 are arranged in an array, which comprises electrodes tailored both for stimulating the nervous system to activate CPM and for stimulating the muscle for muscle contraction. The electrodes may be integrated within a strap to achieve better geometric flexibility. The plurality of electrodes allows customization of treatment in different body locations, and the possibility of automatic correction of stimulus placement in cases where the placement is not precise enough. For some applications, the computer processor is configured to adaptively control which electrodes are active based on location selection and/or feedback from an accelerometer, as described hereinbelow.

For some applications, the computer processor is configured to select which electrodes in the electrode array apply the CPM signal and which electrodes apply the muscle contraction signal. Optionally, the same electrodes are used to apply both signals, in which case the computer processor is configured to generate a combined signal in which, for example, the muscle contraction signal modulates the CPM signal.

The selection of electrodes may be based on the treatment location on the body, as well as information from previous treatments. For some applications, the computer processor automatically changes the electrode selection upon a detection that the muscle is not responding to the electrical stimulus and is not contracting.

For some applications, the intensity of the muscle contraction signal is set based on information from a signal library, the system's position on the body, feedback from the patient, and/or feedback from a muscle contraction detection system. Typically, the computer processor is configured to apply the minimal necessary muscle contraction signal and the maximum possible CPM signal without causing excessive discomfort.

Optionally, the system uses historical information about the optimal treatment intensity for the patient at each location, along with real-time feedback to determine the treatment intensity and the ratio between the stimulus intensity for muscle contraction and the stimulus intensity for CPM activation.

Optionally, a clinical database is populated with the waveforms, modulation, and desired treatment intensity for treating each location based on clinical research. These data may be updated based on user feedback to generate the signals.

For some applications, the computer processor uses data from an accelerometer and, optionally, a machine learning-based decoding model or a corresponding heuristic algorithm, to ascertain whether the intensity and location of the muscle stimulus indeed achieve the goal of producing cyclic contraction at a low frequency.

For some applications, the computer processor is configured to regulate the intensity of the applied signal, by translating the contraction detection into a signal to the processor indicating to the processor that it should increase the muscle stimulus intensity at the expense of the CPM intensity. Optionally, if the signal is amplified several times without response, the system changes the muscle stimulus transmission location, either by changing which electrodes in the electrode array are activated or by sending a message to the user interface to ask the user to adjust the device's location on the arm or leg.

For some applications, a user application and cloud system is provided that includes feedback on the patient's discomfort level (tolerability), indication about changing the device's location on the body (location selection), adjustments of the treatment protocol, and a treatment log that includes a history of treatments, stimulus intensity, and/or patient feedback during and after the treatment regarding the treatment's efficacy and/or side effects, if any. The treatment log is synchronized with a medical cloud system that can be used to optimize the treatment by the caregiver (personal physician) or by an AI algorithm.

For some applications, the apparatus further includes an accelerometer (e.g., a 3D, 6D, or 9D accelerometer) configured to generate an accelerometry signal indicative of motion of the portion of the body of the subject, such as local muscle contraction, e.g., twitching, caused by application of the amplitude shift keying signal, such as to release endorphins. The computer processor is configured to modify a parameter of the amplitude shift keying signal in response to the accelerometry signal.

For some applications, the accelerometer is configured to generate the accelerometry signal in response to contraction of a muscle of the subject.

For some applications, the computer processor is configured to increase an energy level of the amplitude shift keying signal in response to determining that a level of the accelerometry signal is below a threshold.

For some applications, the accelerometry signal is indicative of cyclic contraction of the muscle, and the computer processor is configured to increase an energy level of the amplitude shift keying signal in response to determining that a level of the accelerometry signal is below a threshold.

For some applications, the apparatus further includes an accelerometer configured to generate an accelerometry signal indicative of motion of the portion of the body of the subject, and the computer processor is configured to direct the subject to move the set of two or more electrodes in response to a low level of the accelerometry signal.

For some applications of the present invention, tracking of muscular contractions is a proxy for successful stimulation.

Some applications of the present invention include one or more of the following three mechanisms to support successful muscle stimulation:
muscle stimulation tracking based on an accelerometer and a built-in algorithm that identifies whether the muscle is contracting properly or not;
an adaptive intensity algorithm, which increases or decreases stimulation intensity with the goal to reach consistent muscle contractions with minimal electric stimulation and discomfort; and/or
an improper location alert, if the muscle stimulation is unable to achieve contractions.

For some applications, automatic and efficient activation of the muscle contraction mechanism is achieved by taking into account:
measurement of muscle response and pain relief to activation,
waveforms and frequency,
shapes, sizes, and placement of electrodes on each part of the body,
the most efficient mechanism (minimum energy for contraction, most effective pain relief),
starting treatment with minimum energy,
gradually increasing the energy until contractions are detected (e.g., using an accelerometer),
gradually increasing the modulating signal until muscle contraction is identified (CPM prioritization),
using an electrode array that allows for “mini movements” of the stimulus location,
activating both treatments with a single set of electrodes, and/or
using a variety of modulation forms.

For some applications, addressing the increased sensitivity of fibromyalgia sufferers to touch and stimulation is provided by:
reducing the stimulus for muscle contraction activation to the minimum (Minimal Effective Dose),
activation at low intensity that increases until automatic detection of muscle contraction (accelerometer),
adapting the device for treatment in different locations,
adapting the device for activation on the arm, calf, or thigh,
selecting electrode location based on personal pain sensitivity, and/or
changing the electrode location during treatment.

For some applications, the above-described electrical stimulation signals are used to provide electrical stimulation to a subject suffering from a condition other than fibromyalgia. Furthermore, the scope of the present application includes applying electrical stimulation signals to a subject having signal characteristics as described herein, but via a different type of electrode to those described hereinabove. For example, the stimulation may be applied via implanted electrodes, subcutaneous electrodes, and/or any other type of electrodes configured to electrically stimulate a subject.

Applications of the invention described herein can take the form of a computer program product accessible from a computer-usable or computer-readable medium (e.g., a non-transitory computer-readable medium) providing program code for use by or in connection with a computer or any instruction execution system, such as computer processor 24. For the purpose of this description, a computer-usable or computer readable medium can be any apparatus that can comprise, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Typically, the computer-usable or computer readable medium is a non-transitory computer-usable or computer readable medium.

Examples of a computer-readable medium include a semiconductor or solid state memory, magnetic tape, a removable computer diskette, a random access memory (RAM), a read-only memory (ROM), a rigid magnetic disk and an optical disk. Current examples of optical disks include compact disk-read only memory (CD-ROM), compact disk-read/write (CD-R/W) and DVD. For some applications, cloud storage, and/or storage in a remote server is used.

A data processing system suitable for storing and/or executing program code will include at least one processor (e.g., computer processor 24) coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution. The system can read the inventive instructions on the program storage devices and follow these instructions to execute the methodology of the embodiments of the invention.

Network adapters may be coupled to the processor to enable the processor to become coupled to other processors or remote printers or storage devices through intervening private or public networks. Modems, cable modem and Ethernet cards are just a few of the currently available types of network adapters.

Computer program code for carrying out operations of the present invention may be written in any combination of one or more programming languages, including an object oriented programming language such as Java, Smalltalk, C++ or the like and conventional procedural programming languages, such as the C programming language or similar programming languages.

It will be understood that the methods described herein can be implemented by computer program instructions. These computer program instructions may be provided to a processor of a general purpose computer, special purpose computer, or other programmable data processing apparatus to produce a machine, such that the instructions, which execute via the processor of the computer (e.g., computer processor 24) or other programmable data processing apparatus, create means for implementing the functions/acts specified in the methods described in the present application. These computer program instructions may also be stored in a computer-readable medium (e.g., a non-transitory computer-readable medium) that can direct a computer or other programmable data processing apparatus to function in a particular manner, such that the instructions stored in the computer-readable medium produce an article of manufacture including instruction means which implement the function/act specified in the methods described in the present application. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide processes for implementing the functions/acts specified in the methods described in the present application.

Computer processor 24 and the other computer processors described herein are typically hardware devices programmed with computer program instructions to produce a special purpose computer. For example, when programmed to perform the methods described herein, the computer processor typically acts as a special purpose electrical-stimulation computer processor. Typically, the operations described herein that are performed by computer processors transform the physical state of a memory, which is a real physical article, to have a different magnetic polarity, electrical charge, or the like depending on the technology of the memory that is used.

In an embodiment, techniques and apparatus described in one or more of the following patents and patent applications, which are assigned to the assignee of the present application and incorporated herein by reference, are combined with techniques and apparatus described herein:
U.S. Pat. No. 9,895,533 to Harpak et al.;
U.S. Pat. No. 10,213,602 to Ironi et al.;
U.S. Pat. No. 10,289,594 to Harpak et al.;
U.S. Pat. No. 10,926,090 to Ironi et al.;
U.S. Pat. No. 11,167,135 to Ironi;
U.S. Pat. No. 11,357,980 to Ironi et al.;
US Patent Application Publication 2021/0052884 to Jashek et al.; and/or
U.S. Provisional Application 63/560,904, filed Mar. 4, 2024, including, but not limited to, a description of an experiment conducted on behalf of the inventors.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. An apparatus comprising:
a set of two or more electrodes configured to be placed in electrical contact with a portion of a body of a subject; and
at least one computer processor configured to drive the electrodes to apply an amplitude shift keying signal into the portion of the subject's body, the amplitude shift keying signal containing:
a high frequency component that acts as a carrier wave, the high frequency component having a frequency of between 80 Hz and 120 Hz, and
a low frequency component that acts as a modulating component that modulates the carrier wave, the low frequency component having a frequency of between 1 Hz and 8 Hz,
the computer processor configured to drive the electrodes to apply the amplitude shift keying signal into the portion of the subject's body by applying the low frequency signal such that:
when a pulse of the low frequency signal is active, a current of the amplitude shift keying signal alternates between a nominal maximum and a nominal minimum of the amplitude shift keying signal, and
when a pulse of the low frequency signal is inactive, the current of the amplitude shift keying signal alternates between the nominal maximum minus a modulation factor and the nominal minimum plus the modulation factor, the modulation factor being between 0.05 and 0.15 of the nominal maximum.

2. The apparatus according to claim 1, wherein the computer processor is configured to set the high frequency component to be between 100 Hz and 120 Hz.

3. The apparatus according to claim 1, further comprising an accelerometer configured to generate an accelerometry signal indicative of motion of the portion of the body of the subject, wherein the computer processor is configured to modify a parameter of the amplitude shift keying signal in response to the accelerometry signal.

4. The apparatus according to claim 3, wherein the accelerometer is configured to generate the accelerometry signal in response to contraction of a muscle of the subject.

5. The apparatus according to claim 4, wherein the computer processor is configured to increase an energy level of the amplitude shift keying signal in response to determining that a level of the accelerometry signal is below a threshold.

6. The apparatus according to claim 4, wherein the accelerometry signal is indicative of cyclic contraction of the muscle, and wherein the computer processor is configured to increase an energy level of the amplitude shift keying signal in response to determining that a level of the accelerometry signal is below a threshold.

7. The apparatus according to claim 1, further comprising an accelerometer configured to generate an accelerometry signal indicative of motion of the portion of the body of the subject, wherein the computer processor is configured to direct the subject to move the set of two or more electrodes in response to a low level of the accelerometry signal.

8. The apparatus according to claim 1,
wherein the set of two or more electrodes is a first set of two or more electrodes,
wherein the apparatus further comprises a second set of two or more electrodes, and an accelerometer configured to generate an accelerometry signal indicative of motion of the portion of the body of the subject, and
wherein the computer processor is configured to drive the electrodes of the second set, rather than the electrodes of the first set, to apply the amplitude shift keying signal in response to a low level of the accelerometry signal.

9. The apparatus according to claim 1, further comprising a patch, wherein the electrodes are disposed upon the patch and the electrodes are configured to be placed in electrical contact with the portion of the subject's body by placing the patch upon the portion of the subject's body.

10. The apparatus according to claim 1, further comprising a mount selected from the group consisting of: a wristwatch, a cuff, and a bracelet, and wherein the electrodes are disposed upon the selected mount and are configured to be placed in electrical contact with the portion of the subject's body by placing the selected mount upon the portion of the subject's body.

11. The apparatus according to claim 1, wherein the computer processor is configured to drive the electrodes to apply the amplitude shift keying signal into the portion of the subject's body by:
applying the high frequency component, the high frequency component including a biphasic pulse, and
applying the low frequency component, the low frequency component including a monophasic pulse.

12. The apparatus according to claim 1, wherein the computer processor is configured to drive the electrodes to apply the amplitude shift keying signal into the portion of the subject's body by applying the high frequency component, the high frequency component having a base frequency, and the frequency of the high frequency component drifting from the base frequency up to 20 percent above the base frequency, and down to 20 percent below the base frequency.

13. A method comprising:
applying an electrical amplitude shift keying signal to a portion of a body of a subject, via electrodes, the amplitude shift keying signal containing:
a high frequency component that acts as a carrier wave, the high frequency component having a frequency of between 80 Hz and 120 Hz, and
a low frequency component that acts as a modulating component that modulates the carrier wave, the low frequency component having a frequency of between 1 Hz and 8 Hz,
wherein applying the electrical amplitude shift keying signal to the portion of the subject's body comprises applying the low frequency signal such that:
when a pulse of the low frequency signal is active, a current of the amplitude shift keying signal alternates between a nominal maximum and a nominal minimum of the amplitude shift keying signal, and
when a pulse of the low frequency signal is inactive, the current of the amplitude shift keying signal alternates between the nominal maximum minus a modulation factor and the nominal minimum plus the modulation factor, the modulation factor being between 0.05 and 0.15 of the nominal maximum.

14. The method according to claim 13, wherein applying the electrical amplitude shift keying signal comprises applying the electrical amplitude shift keying signal to a portion of a body of a subject who has fibromyalgia.

15. The method according to claim 13, wherein applying the electrical amplitude shift keying signal to the portion of the subject's body comprises:
applying the high frequency component, the high frequency component including a biphasic pulse, and
applying the low frequency component, the low frequency component including a monophasic pulse.

16. The method according to claim 13, wherein applying the electrical amplitude shift keying signal to the portion of the subject's body comprises applying the high frequency component, the high frequency component having a base frequency, and the frequency of the high frequency component drifting from the base frequency up to 20 percent above the base frequency, and down to 20 percent below the base frequency.

17. The method according to claim 13, wherein applying the electrical amplitude shift keying signal comprises setting the high frequency component to be between 100 Hz and 120 Hz.

18. The method according to claim 13, wherein applying the electrical amplitude shift keying signal further comprises modifying a parameter of the amplitude shift keying signal in response to an accelerometry signal generated by an accelerometer and indicative of motion of the portion of the body of the subject.

19. The method according to claim 18, wherein the accelerometer is configured to generate the accelerometry signal in response to contraction of a muscle of the subject.

20. The method according to claim 19, wherein applying the electrical amplitude shift keying signal comprises increasing an energy level of the amplitude shift keying signal in response to determining that a level of the accelerometry signal is below a threshold.

21. The method according to claim 19, wherein the accelerometry signal is indicative of cyclic contraction of the muscle, and wherein applying the electrical amplitude shift keying signal comprises increasing an energy level of the amplitude shift keying signal in response to determining that a level of the accelerometry signal is below a threshold.

22. The method according to claim 13, further comprising directing the subject to move the electrodes in response to a low level of an accelerometry signal generated by an accelerometer and indicative of motion of the portion of the body of the subject.

23. The method according to claim 13,
wherein the electrodes are first electrodes, and
wherein applying the electrical amplitude shift keying signal comprises applying the amplitude shift keying signal via second electrodes, rather than the first electrodes, in response to a low level of an accelerometry signal generated by an accelerometer and indicative of motion of the portion of the body of the subject.
